(19) United States
(12) Patent Application Publication
(10) Pub. No.: US 2026/0165681 A1
(43) Pub. Date: Jun. 18, 2026

(54) MEDICAL PHANTOM COMPRISING AN ECHOGENIC AND RADIOPAQUE BONE-MIMICKING PRODUCT

(71) Applicant: INSTITUT NATIONAL DE RECHERCHE EN INFORMATIQUE ET EN AUTOMATIQUE, LE CHESNAY (FR)

(72) Inventor: Tomáš SVATON, LE CHESNAY (FR)

(21) Appl. No.: 19/126,416

(22) PCT Filed: Oct. 31, 2023
(86) PCT No.: PCT/EP2023/080337

§ 371 Date: May 1, 2025

(30) Foreign Application Priority Data
Nov. 2, 2022 (FR) FR2211420

(51) Int. Cl. A61B 8/00; A61B 6/00; A61B 6/58; A61B 8/06

(52) U.S. Cl. CPC A61B 8/5207; A61B 6/481; A61B 6/583; A61B 8/06; A61B 8/4444; A61B 8/483

(57) ABSTRACT

A medical phantom suitable for ultrasound scan and radiography training including a silicone dummy within which is arranged a product for mimicking at least one bone. The product is formed from a polymer doped with a radiopaque agent according to either of the compositions below: in a first composition, the polymer is polylactic acid and the radiopaque agent is selected from copper, stainless steel and brass, in a second composition, the polymer is an epoxy resin or a polyurethane resin and is mixed with a hardener, the radiopaque agent being barium sulphate.

### CROSS-REFERENCE TO RELATED APPLICATIONS

This Application is a Section 371 National Stage Application of International Application No. PCT/EP2023/080337, filed Oct. 31, 2023, and published as WO 2024/094683 A1 on May 10, 2024, not in English, which claims priority to and the benefit of French Patent Application No. 2211420, filed Nov. 2, 2022, the contents of which are incorporated herein by reference in their entireties.

### FIELD OF THE INVENTION

The field of the invention relates to a medical phantom comprising an echogenic and radiopaque bone-mimicking product. Such a medical phantom is particularly suitable for training in medical imaging techniques, particularly ultrasound scan and radiography.

### BACKGROUND

Ultrasound scan is a medical imaging technique that makes it possible, thanks to the analysis of echoes produced by ultrasounds on internal tissues, to morphologically explore anatomical structures.

Ultrasound scan requires the use of a probe for transmitting and receiving ultrasounds. The probe generally comprises a piezoelectric ceramic which, when subjected to electrical pulses, generates ultrasounds. The probe is brought into contact with the skin and the emitted ultrasounds pass through the tissues, more or less deeply depending on the frequency thereof, then are returned in the form of echoes. Computer processing subsequently makes it possible to convert the echoes received into images of the explored anatomical region. A gel can be applied to amplify the transmission of ultrasounds and improve the quality of the obtained images.

Ultrasound scan also makes it possible to diagnose abnormalities, diseases or pathologies affecting organs as well as to monitor the progress of a pregnancy. Ultrasound scan is also used in rheumatology for the intra-articular injection of a drug—or infiltration—using a needle to relieve a painful joint. Moreover, it is common for a healthcare professional to use ultrasound scan during a transcutaneous biopsy to guide the needle.

Radiography is a medical imaging technique that involves exposing an anatomical region to X-rays to generate therefrom a greyscale image whereon anatomical structures can be visualised by contrast depending on the respective attenuation coefficients thereof.

The X-rays are produced by an X-ray tube formed by a vacuum enclosure within which are arranged a cathode and an anode. An electric current is applied to the cathode to heat it while a potential difference is generated between the cathode and the anode. An electron beam is emitted from the cathode to the anode and is accelerated by the potential difference. The anode restores the energy supplied by the electrons partly in the form of X-rays. The X-rays subsequently pass through anatomical structures that differ in the thickness thereof and the attenuation coefficient thereof. Finally, a detector converts the captured photons exiting the anatomical region into a greyscale image. A contrast agent can be injected to improve visibility.

Radiography makes it possible to visualise muscles, joints and especially bones that indeed have a higher attenuation coefficient than soft tissues. This is then referred to as radiopacity—or radiodensity—that is to say the ability to oppose the passage of X-rays. Radiography is used to diagnose cracks and fractures or to detect a foreign body.

Mastering these medical imaging techniques requires training. Ultrasound scan guidance training medical phantoms currently exist to practise handling the probe, and possibly the needle. Moreover, radiography guidance training medical phantoms make it possible to learn how to use the X-ray tube and the detector as well as to interpret the obtained image.

However, no known medical phantom is suitable for both ultrasound scan training and radiography training.

### SUMMARY

The present invention improves the situation.

To this end, the invention relates to a medical phantom comprising a silicone dummy within which is arranged a product mimicking at least one bone.

The product is formed from a polymer doped with a radiopaque agent according to:
a first composition wherein the polymer is polylactic acid, the radiopaque agent being selected from copper, stainless steel and brass, or
a second composition wherein the polymer is an epoxy resin or a polyurethane resin and is mixed with a hardener, the radiopaque agent being barium sulphate.

In one or more embodiments, the product is formed according to the first composition wherein polylactic acid is doped with copper at a doping level between 14 and 20%, and preferably substantially equal to 18%.

According to an alternative embodiment, the product is formed according to the first composition wherein polylactic acid is doped with stainless steel at a doping level between 13 and 27%, and preferably substantially equal to 21%.

According to another alternative embodiment, the product is formed according to the first composition wherein polylactic acid is doped with brass at a doping level between 14 and 28%, and preferably substantially equal to 23%.

In one or more embodiments, the product is formed according to the second composition wherein the polymer is an epoxy resin and is doped with barium sulphate at a doping level between 5 and 18%, and preferably substantially equal to 12%.

According to one alternative embodiment, the epoxy resin is resoltech 1050 resin and the hardener is of the 105xS type.

According to another alternative embodiment, the epoxy resin is SR GreenPoxy 56 resin and the hardener is SD 7561.

According to another alternative embodiment, the epoxy resin is CHS-epoxy resin 324 and the hardener is P11.

In one or more embodiments, the product is formed according to the second composition wherein the polymer is a polyurethane resin and is doped with barium sulphate at a doping level between 20 and 50%, and preferably substantially equal to 33%.

For example, the polyurethane resin is a resin from the Formousse range and the hardener is of the MD type.

The invention also relates to a method for manufacturing a medical phantom as described above.

The method comprises the following operations of:
manufacturing the product from the first composition or from the second composition,
superimposing a first negative mould comprising an impression of the shape of a front face of the dummy to be obtained and a positive mould comprising a relief of the shape of the product,
pouring silicone between the first negative mould and the positive mould,
removing the positive mould after hardening the silicone, the positive mould leaving an impression of the shape of the product in the silicone,
placing the product in the first negative mould in the impression left by the positive mould,
superimposing the first negative mould and a second negative mould comprising an impression of the shape of a rear face of the dummy to be obtained,
pouring silicone between the first negative mould and the second negative mould,
removing the first negative mould and the second negative mould after hardening the silicone to obtain the dummy within which is arranged the product.

In one or more embodiments, the product is manufactured by extrusion three-dimensional printing from the first composition.

In one or more embodiments, the product is manufactured by pouring the second composition in liquid form into an impression of a mould.

### BRIEF DESCRIPTION OF THE DRAWINGS

Other features, details and advantages will become apparent upon reading the detailed description hereinafter, and from the analysis of the appended drawings, wherein:

FIG. 1 schematically illustrates a medical phantom according to the invention;

FIG. 2 illustrates a medical phantom according to the invention comprising a dummy of a human foot;

FIG. 3 illustrates a product for mimicking a spine of a medical phantom according to the invention;

FIG. 4 illustrates images, according to a sectional view, obtained by ultrasound scan, respectively of a medical phantom according to the invention mimicking a human hand and of a real human hand;

FIG. 5 illustrates an image, according to a top view, obtained by radiography of a medical phantom according to the invention mimicking a human hand and of a real human hand;

FIG. 6 illustrates features of the attenuation of a detector used in radiography;

FIG. 7 illustrates a method for manufacturing a medical phantom according to the invention;

FIG. 8 illustrates moulds used during the method of [FIG. 7]; and

FIG. 9 illustrates a pouring operation of the method of [FIG. 7] involving the moulds of [FIG. 8].

### DETAILED DESCRIPTION OF ILLUSTRATIVE EMBODIMENTS

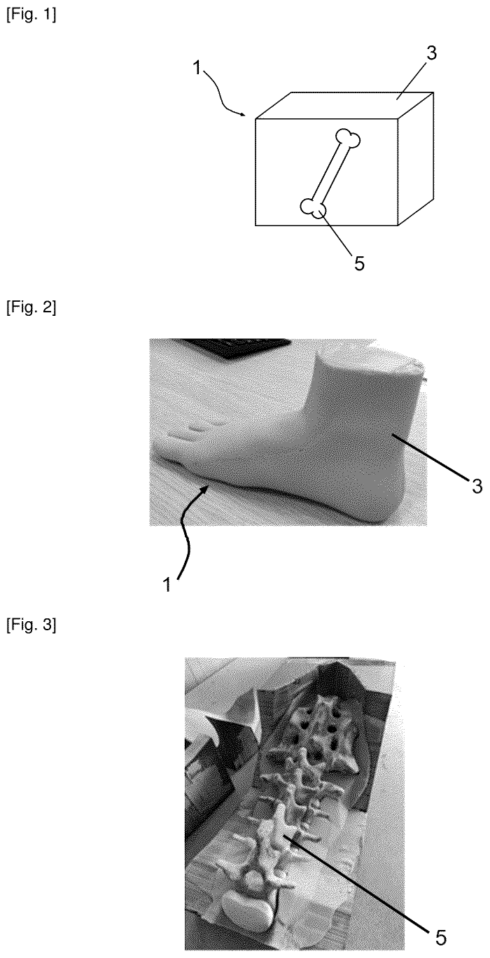

FIG. 1 schematically illustrates a medical phantom 1.

The medical phantom 1 may make it possible for a healthcare professional, for example a radiologist, to practise ultrasound scan and radiography guidance without having to use different training devices. For such use, the medical phantom 1 may be qualified as a procedural medical simulator.

Ultrasound scan training generally involves the handling of a probe to be applied to the skin to visualise an anatomical region. Such training may include the application of a gel to amplify the transmission of ultrasound scans as well as the handling, in conjunction with the probe, of a needle that injects an anaesthetic product—usually cortisone—into a joint or performs a transcutaneous biopsy. Ultrasound scan makes it possible to guide the needle more accurately.

The technique of radiography requires the mastery of various tools, particularly an X-ray tube and a detector. In particular, prolonged exposure to X-rays, which are ionising electromagnetic radiation, may pose a risk to the health of the patient such that the X-ray tube must be handled with care. The detector provides a greyscale image of an anatomical region and requires practice to obtain an image of sufficient quality and to be able to interpret it. Moreover, the injection of a contrast agent, sometimes necessary to visualise soft tissues or organs, may also be subject to training.

It should be noted that the medical phantom 1 is not assigned to ultrasound scan and radiography training alone. The properties thereof, detailed in the following description, make it possible for it to be used for training in other medical imaging techniques or even non-medical uses.

As illustrated in [FIG. 1], the medical phantom 1 comprises a silicone dummy 3 within which a product 5 for mimicking at least one bone is arranged.

The dummy 3 is adapted to mimick a part of the human body. To this end, silicone has properties similar to those of soft tissue in the presence of ultrasounds or X-rays. Moreover, silicone has the advantage of having self-healing properties, which is useful in case of the formation of orifices by the repeated use of a needle.

Advantageously, the dummy 3 has an external shape similar to that of the part of the body that it is intended to mimick by ultrasound scan and radiography. Indeed, such a shape makes it possible to increase the realism of the medical phantom 1 and to make the training as similar as possible to a real examination.

For example, [FIG. 2] illustrates one embodiment of the medical phantom 1 wherein the dummy 3 takes the shape of a human foot. The medical phantom 1 shown here makes it possible for a healthcare professional to practice ultrasound scan for the detection of plantar fasciitis or tendonitis or radiography to assess heel positioning when pressing thereon or diagnose a fracture of the metatarsus. The product 5, arranged within the dummy 3, is not visible in [FIG. 2].

However, the dummy 3 may also be of any shape, in which case the dummy 3 mainly makes it possible to mimick the soft tissues that surround and hold the skeleton.

The product 5 is adapted to mimick a bone and, more generally, a part of the human skeleton. As opposed to the dummy 3, which may have any external shape, the external shape of the product 5 is advantageously similar to that of the bone or of the part of the skeleton to be mimicked.

In the example illustrated in [FIG. 3], the product 5 takes the form of a human spine—or spinal column. The product 5 then comprises a plurality of dummy bones so as to mimick, among other things, the cervical spine, the dorsal spine, the lumbar spine, the sacrum and the coccyx. The product 5 shown in [FIG. 3] is intended to be arranged in a silicone dummy 3 advantageously taking the form of a human thorax to form a medical phantom 1. Such a medical phantom 1 makes it possible for a healthcare professional to practice ultrasound scan for identification before an epidural infiltration or radiography to look for a static disorder such as scoliosis or kyphosis.

Bones are rigid structures, essentially consisting of connective tissue, which together form the skeleton. Bones make it possible to protect internal organs and promote movement. They are involved in calcium metabolism, mineral storage and blood cell production.

The constitution of a bone gives it specific properties in the presence of ultrasounds or X-rays that make it visible with ultrasound scan and radiography.

First of all, bones are hyperechoic solid structures. During an ultrasound scan, the bones reflect the ultrasounds back to the probe in the form of echoes. This property makes it possible, for example, to detect an irregularity of the cortical hyperechoic line, which suggests a fracture or the avulsion of an enthesis, as well as to diagnose then puncture subperiosteal haematic collections.

Moreover, bones have a higher attenuation coefficient than that of soft tissues or organs and thus hinder the passage of photons that make up X-rays. This property—radiopacity—makes it possible for bones to be visible in the greyscale image obtained by radiography. It is thus possible to observe, at the bone level, a fracture, a bone deviation, an infection or a tumour and, at the joint level, osteoarthritis, an accumulation of fluid in a joint (effusion) or a dislocation.

Consequently, the inventor has oriented their research on compositions that make it possible to manufacture dummy bones, here the product 5, the properties of which are similar to those of real bones both with ultrasound scan and radiography. The results of the work of the inventor are detailed hereinafter and relate to compositions wherein the product 5 is formed from a polymer doped with a radiopaque agent.

The inventor found that polylactic acid (better known by the acronym PLA), epoxy resin and polyurethane resin make it possible for the product 5 to reflect ultrasounds in a manner similar to that of bones and thus offer the sought visibility with ultrasound scan.

The epoxy resin is, for example, resoltech 1050 resin marketed by Résoltech (registered trademark). Resoltech 1050 resin can be mixed with a 105xS type hardener to form an epoxy polymer—or polyepoxide. The recommended dosage is 35 parts of 105xS hardener per 100 parts of resoltech 1050 resin by weight. Moreover, it is possible to mix these hardeners to adjust the hardening speed. It should be noted that the mixing can be carried out at room temperature, that is to say between 18 and 25° C., and that a post-baking is not necessary for demoulding.

In an alternative embodiment, the SR GreenPoxy 56 resin marketed by Sicomin may be used. GreenPoxy 56 SR resin can be mixed with the SD 7561 hardener to form an epoxy polymer. The recommended dosage is 36 parts of SD 7561 hardener per 100 parts of GreenPoxy 56 SR resin by weight. Here again, the mixing can be carried out at ambient temperature.

Also in an alternative embodiment, CHS-epoxy 324 resin marketed by Spolchemie (registered trademark) can be used. CHS-epoxy resin 324 can be mixed with the P11 hardener to form an epoxy polymer. The recommended dosage is 7 parts of P11 hardener per 100 parts of CHS-epoxy resin 324 by weight.

As regards polyurethane resin, it is possible to use the Formousse range marketed by COP (registered trademark), for example Formousse 60 or Formousse 200 resin. The polyurethane resin of the Formousse range can be mixed with the MD hardener which is a liquid polymeric isocyanate of the MDI type. The recommended dosage is 100 parts of MD hardener per 100 parts of polyurethane resin from the Formousse range by weight.

In the following description, a first composition wherein the polymer is polylactic acid and a second composition wherein the polymer is an epoxy resin or a polyurethane resin are distinguished for the product 5.

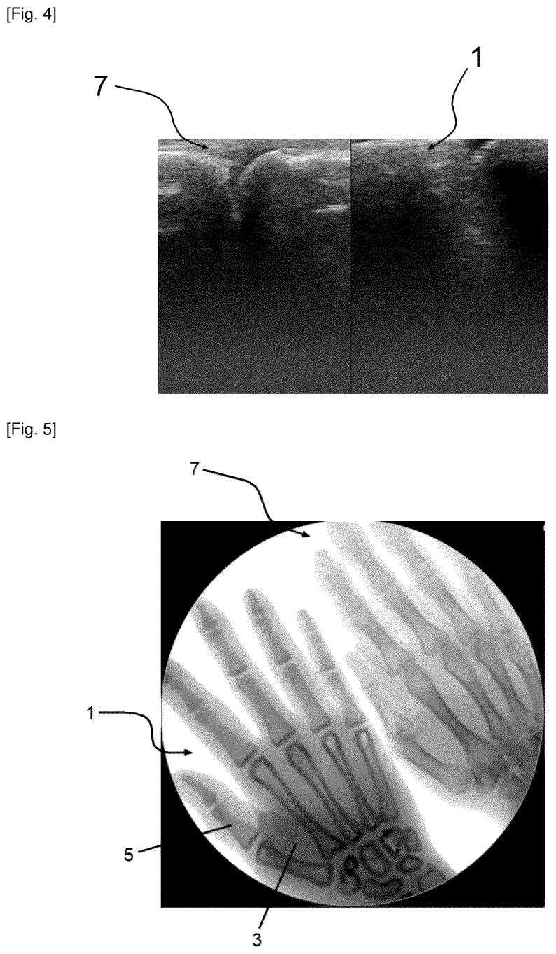

FIG. 4 illustrates two images obtained by ultrasound scan: the image on the right is that of a medical phantom 1 mimicking a human hand while the image on the left is that of a real human hand 7. More precisely, these images make it possible to visualise, according to a sectional view, the metacarpus which is therefore mimicked, in the medical phantom 1, by a product 5. The comparison of these two images highlights that the product 5 offers a satisfactory visual and therefore makes it possible to practice ultrasound scan guidance.

The inventor subsequently tested various radiopaque agents to be mixed with the polymer to obtain a product 5 suitable for impeding the passage of photons and being characterized by a high attenuation coefficient to offer the sought visibility with radiography.

First of all, as regards the first composition of the product 5, that is to say that wherein the polymer is polylactic acid, the inventor established that copper, stainless steel and brass make it possible to confer the sought radiopacity on polylactic acid.

In particular, polylactic acid can be doped with copper at a doping level advantageously between 14 and 20%. Preferably, the doping level is substantially equal to 18%.

In an alternative embodiment, polylactic acid can be doped with stainless steel at a doping level advantageously between 13 and 27%. Preferably, the doping level is substantially equal to 21%.

Also in an alternative embodiment, polylactic acid may be doped with brass at a doping level advantageously between 14 and 28%. Preferably, the doping level is substantially equal to 23%.

The doping level here refers to the ratio of the weight of radiopaque agent to the total weight of the first composition, therefore the weight of polylactic acid and of radiopaque agent:

$$τ = \frac{{m}_{aro}}{{m}_{PLA} + {m}_{aro}}$$

where:—τ is the doping level,
mₐᵣₒ is the weight of radiopaque agent, and
m_{PLA} is the weight of polylactic acid.

“Substantially equal” means here that, ideally, polylactic acid is doped with the radiopaque agent—copper, stainless steel or brass—at the indicated doping level. However, in practice, it is difficult or even impossible to obtain exactly the desired doping level, such that the doping level may deviate by 1% from the target doping level.

Now as regards the second composition of the product 5, that is to say that wherein the polymer is epoxy resin or polyurethane resin, the inventor established that barium sulphate (also designated by the chemical formula BaSO₄) makes it possible to confer the sought radiopacity on the selected resin.

In particular, the epoxy resin may be doped with barium sulphate at a doping level advantageously between 5 and 18%. Preferably, the doping level is substantially equal to 12%.

In more detail, the doping level of barium sulphate is advantageously between 5 and 18% for resoltech 1050 resin and between 6 and 17% for GreenPoxy 56 SR resin.

As regards the polyurethane resin, and more particularly the Formousse 60 or Formousse 200 resin, the doping level of the barium sulphate is advantageously between 20 and 50%. Preferably, the doping level is substantially equal to 33%.

The doping level here refers to the ratio of the weight of radiopaque agent, therefore barium sulphate, to the total weight of the second composition, therefore the weight of resin, hardener and radiopaque agent:

$$τ = \frac{{m}_{aro}}{{m}_{res} + {m}_{hard} + {m}_{aro}}$$

where:—τ is the doping level,
mₐᵣₒ is the weight of radiopaque agent,
mᵣₑₛ is the weight of resin, and
m_{hard} is the weight of hardener.

“Substantially equal” means here that, ideally, the resin is doped with barium sulphate at the indicated doping level. However, in practice, it is difficult or even impossible to obtain exactly the desired doping level, such that the doping level may deviate by 1% from the target doping level.

By way of illustration, [FIG. 5] illustrates, on the same image obtained by radiography, a medical phantom 1 mimicking a human hand and a real human hand 7. Here again, the product 5 mimics the bones of the hand and is arranged in a silicone dummy 3 the external shape of which is that of a human hand. The comparison with the real human hand 7 makes it possible to verify that the medical phantom 1 thus obtained is visually very similar to the part of the body—in this case a hand—to be mimicked and therefore offers the guarantees necessary for radiography guidance training.

To determine the appropriate doping level, the inventor used a detector usually used in radiography. Such a detector is for example an inorganic scintillator comprising crystals and a photomultiplier tube (better known by the acronym PMT). The crystals are disposed in an array configuration and each comprises scintillation material, generally sodium iodide, which, in response to X-ray absorption, emits photons in the visible range. The photomultiplier tube exploits the photoelectric effect to convert the light received from the crystal array into an electrical signal.

The detector converts the received photons into a greyscale image. The greyscale of a given point of the image depends on the intensity I of the X-ray beam received by the corresponding crystal of the detector. The intensity I of the X-ray beam after passing through a material depends on the incident intensity I₀ and the attenuation coefficient μ of the material according to the following exponential attenuation law:

$$I = {I}_{0} {e}^{- μ ( x )}$$

where: the attenuation coefficient μ of the material is a function of the thickness x of the material passed through.

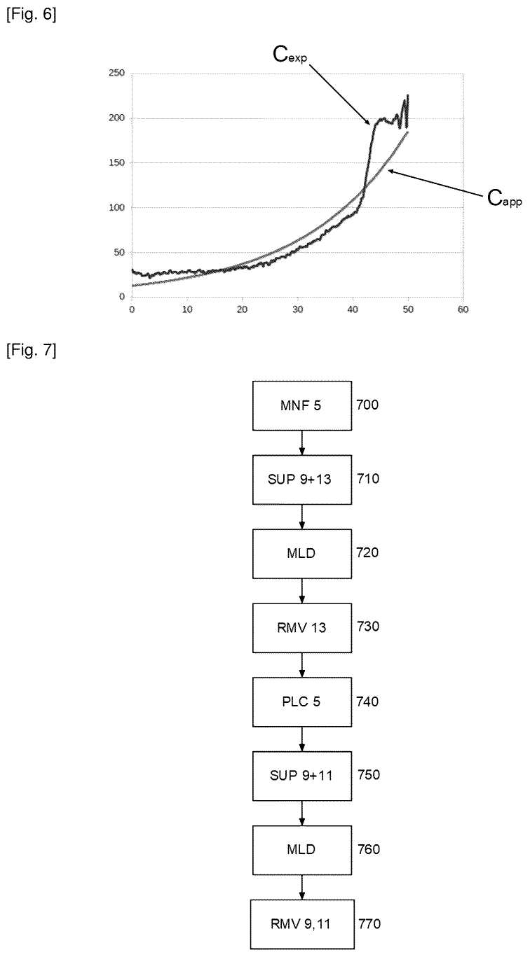

The operation of a detector can be characterised by the function that associates an attenuation coefficient with a given thickness of a material. Such a function—also known as a feature—is shown in [FIG. 6]. More precisely, [FIG. 6] illustrates a curve Cₑₓₚ that corresponds to the experimental feature Cₑₓₚ of a detector and a curve Cₐₚₚ that corresponds to an approximation of the experimental feature Cₑₓₚ. The thickness on the x-axis is in millimetres (mm) while the attenuation coefficient on the y-axis is without units.

Consequently, on an image obtained by radiography, the difference in greyscale between two points indicates a difference in attenuation coefficient. In particular, the contrast of such an image results from the difference in attenuation coefficient between bones and other tissues. The ratio of the attenuation coefficient of bones to the attenuation coefficient of other tissues is approximately 2.5. To evaluate the quality of a composition, that is to say a polymer doped with a radiopaque agent, the inventor manufactures a cube of the composition to be tested and carries out an X-ray to obtain, on the same image, the manufactured cube and a silicone cube. The contrast between the two cubes on the obtained image makes it possible to deduce therefrom the ratio of the coefficient of attenuation of the composition tested to the attenuation coefficient of the silicone, the quality of the composition being all the more satisfactory as the measured contrast corresponds to a ratio in the order of 2.5.

A method for manufacturing the medical phantom 1 will now be described with reference to [FIG. 7].

The method can be broken down into two phases. The first phase, corresponding to operation 700, is a manufacturing phase of the product 5. The second phase, corresponding to operations 710, 720, 730, 740, 750, 760 and 770, is a phase of manufacturing the dummy 3 around the product 5.

During operation 700, the product 5 is manufactured from a polymer doped with a radiopaque agent according to the first composition or the second composition.

First of all, the case where the first composition is selected is considered. As detailed above, the first composition has the particular feature that the polymer is polylactic acid and the radiopaque agent is copper, stainless steel or brass.

The product 5 can then be manufactured by extrusion three-dimensional printing—also called 3D printing or additive manufacturing (also referred to as Material Extrusion). More precisely, extrusion is carried out by fused deposition modelling (better known by the acronym FDM).

This technique consists in feeding an extrusion head—or extruder—of a 3D printer with polylactic acid and the radiopaque agent. Conventionally, the extrusion head can be fed by a coil around which the first composition is wound in the form of a filament. However, it is also possible to introduce polylactic acid and the radiopaque agent in the form of granules into the extrusion head. The extrusion head is heated to melt the first composition and then deposit it, via an extrusion nozzle, on a printing platform. The first composition is thus deposited layer by layer so as to obtain the product 5. Indeed, an operator can programme the 3D printer to obtain the shape of their choice, namely a bone or a part of the skeleton to mimick. To improve the adhesion of successive layers, the printing platform can be heated.

Three-dimensional printing makes it possible to obtain a hollow product 5 which, visually, is very similar to the bone or to the part of the skeleton to be mimicked.

The case in which the second composition is selected is subsequently considered. As detailed above, the second composition has the particular feature that the polymer is an epoxy resin or a polyurethane resin and is mixed with a hardener, while the radiopaque agent is barium sulphate.

The product 5 can then be manufactured by pouring according to a known method.

Typically, a model of the bone or of the part of the skeleton to be mimicked is placed in a boxing having an opening. The model can be held, to facilitate the subsequent removal of the model, by a rod extending outside of the boxing. Silicone is subsequently poured into the boxing through the opening until it covers the model. After hardening (for example vulcanisation type polymerisation) the silicone, the boxing is removed as well as the model, for example by using the rod provided for this purpose. A silicone mould is then obtained the impression of which, that is to say the cavity left by the model in the silicone, has the shape of the bone or of the part of the skeleton to be mimicked. Finally, the doped resin is poured in liquid form into the impression to obtain, after hardening of the doped resin and removal from the mould, the product 5.

In the particular case where the resin used is polyurethane resin, it can be sprayed into the impression using a foam gun, particularly to better control the expansion of the polyurethane resin that follows spraying.

Once the product 5 has been obtained, the phase of manufacturing the dummy 3 is implemented. The dummy 3 is formed so as to envelop the product 5, in the same way that the soft tissues of the human body envelop the skeleton.

To do this, two negative moulds are used: a first negative mould 9 and a second negative mould 11 each having an impression the shape of which is respectively that of the front face and that of the rear face of the dummy 3 to be manufactured. Finally, a positive mould 13 is also used and has a relief the shape of which is, at least partially, that of the product 5 intended to be arranged within the dummy 3.

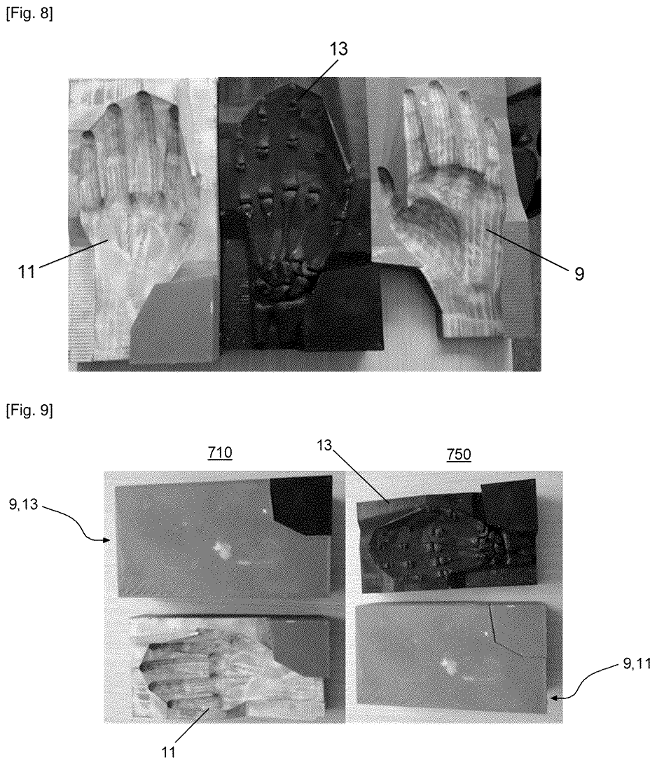

In the example illustrated in [FIG. 8], the dummy 3 to be obtained is in the shape of a hand. Consequently, the first negative mould 9 has an impression of the shape of the palmar face—or front face of the hand-while the second negative mould 11 has an impression of the shape of the dorsal face—or rear face of the hand. The relief of the positive mould 13 has the shape of all of the bones of the hand—carpus, metacarpus and phalanges. A product 5 (not shown here) mimicking all of the bones of the hand was previously manufactured.

As mentioned above, the dummy 3 does not necessarily have the external shape of a part of the body, in which case the front face and the rear face of the dummy 3 to be manufactured are of any kind.

During operation 710, the first negative mould 9 and the positive mould 13 are superposed so as to form a cavity delimited, on the one hand, by the impression of the first negative mould 9 and, on the other hand, by the relief of the positive mould 13.

This operation is illustrated in [FIG. 9] whereon the first negative mould 9 and the positive mould 13 are fitted. The second negative mould 11 is for the moment left aside.

During operation 720, silicone is poured between the first negative mould 9 and the positive mould 13. In other words, the silicone is poured into the cavity formed by the first negative mould 9 and the positive mould 13.

During operation 730, the positive mould 13 is removed after hardening (for example vulcanisation type polymerisation) the silicone. The cavity of the first negative mould 9 is then filled with silicone. Furthermore, the outer surface of the silicone, that is to say the surface which is not in direct contact with the first negative mould 9, has an impression formed and left by the positive mould 13.

During operation 740, the product 5 is placed in the cavity left by the positive mould 13 in the silicone.

During operation 750, the first negative mould 9 and the second negative mould 11 are superposed. It is understood that the product 5 is then taken between the first negative mould 9 and the second negative mould 11.

The superposition of the first negative mould 9 and of the second negative mould 11 forms a cavity delimited, on the one hand, by the outer surface of the silicone the impression of which receives the product 5 and, on the other hand, by the impression of the second negative mould 11.

This operation is illustrated in [FIG. 9] whereon the first negative mould 9 and the second negative mould 11 are fitted, thus enclosing the product 5. The positive mould 13 has been removed and is left aside.

During operation 760, silicone is poured between the first negative mould 9 and the second negative mould 11. In other words, the silicone is poured into the cavity formed by the first negative mould 9 and the second negative mould 11 and covers the product 5 therein.

Finally, during operation 770, the first negative mould 9 and the second negative mould 11 are removed after hardening (for example vulcanisation type polymerisation) the silicone. The medical phantom 1 is then obtained comprising the dummy 3 within which the product 5 is arranged.

In the foregoing, the medical phantom 1 is presented as an ultrasound scan and radiography training tool mimicking a part of the human body, particularly by the shape of the product 5 that is as similar as possible to that of a human bone or of a part of the human skeleton and possibly by the shape of the dummy 3. However, the person skilled in the art understands that the medical phantom 1 can also be adapted to mimick a part of the body of an animal of the vertebrate subbranch and thus be intended for a veterinarian.

Although the present disclosure has been described with reference to one or more examples, workers skilled in the art will recognize that changes may be made in form and detail without departing from the scope of the disclosure and/or the appended claims.

1. A medical phantom comprising:
a silicone dummy within which is arranged a product mimicking at least one bone, wherein the product is formed from a polymer doped with a radiopaque agent according to:
a first composition wherein the polymer is polylactic acid, the radiopaque agent being selected from copper, stainless steel or brass, or
a second composition wherein the polymer is an epoxy resin or a polyurethane resin and is mixed with a hardener, the radiopaque agent being barium sulphate.

2. The medical phantom according to claim 1, wherein the product is formed according to the first composition wherein the polylactic acid is doped with copper at a doping level between 14% and 20%, and preferably substantially equal to 18%.

3. The medical phantom according to claim 1, wherein the product is formed according to the first composition wherein the polylactic acid is doped with stainless steel at a doping level between 13% and 27%, and preferably substantially equal to 21%.

4. The medical phantom according to claim 1, wherein the product is formed according to the first composition wherein the polylactic acid is doped with brass at a doping level between 14% and 28%, and preferably substantially equal to 23%.

5. The medical phantom according to claim 1, wherein the product is formed according to the second composition wherein the polymer is an epoxy resin and is doped with barium sulphate at a doping level between 5% and 18%, and preferably substantially equal to 12%.

6. The medical phantom according to claim 5, wherein the epoxy resin is resoltech 1050 resin and the hardener is of the 105xS type.

7. The medical phantom according to claim 5, wherein the epoxy resin is SR GreenPoxy 56 resin and the hardener is SD 7561.

8. The medical phantom according to claim 5, wherein the epoxy resin is CHS-EPOXY resin 324 and the hardener is P11.

9. The medical phantom according to claim 1, wherein the product is formed according to the second composition wherein the polymer is a polyurethane resin and is doped with barium sulphate at a doping level between 20% and 50%, and preferably substantially equal to 33%.

10. The medical phantom according to claim 9, wherein the polyurethane resin is a resin from the Formousse range and the hardener is of the MD type.

11. A method for manufacturing a medical phantom according to claim 1, said method comprising the following operations of:
manufacturing the product from the first composition or from the second composition,
superimposing a first negative mould comprising an impression of a shape of a front face of the dummy to be obtained and a positive mould comprising a relief of a shape of the product,
pouring the silicone between the first negative mould and the positive mould,
removing the positive mould after hardening the silicone, said positive mould leaving an impression of the shape of the product in the silicone,
placing the product in the first negative mould in the impression left by the positive mould,
superimposing the first negative mould and a second negative mould comprising an impression of the shape of a rear face of the dummy be obtained,
pouring the silicone between the first negative mould and the second negative mould,
removing the first negative mould and the second negative mould after hardening the silicone to obtain the dummy within which is arranged the product.

12. The method according to claim 11, wherein the product is manufactured by extrusion three-dimensional printing from the first composition.

13. The method according to claim 11, wherein the product is manufactured by pouring the second composition in liquid form into an impression of a mould.
